(19) United States
(12) Patent Application Publication
(10) Pub. No.: US 2008/0261249 A1
(43) Pub. Date: Oct. 23, 2008

(54) Detecting and Quantifying Host Cell Proteins in Recombinant Protein Products

(75) Inventors: Yajun "Jennifer" Wang, Pearland, TX (US); Meng-Yuan "Patrick" Liu, Pearland, TX (US)

Correspondence Address: Nikolaos C. George;JONES DAY, 222 E. 41st. Street, New York, NY, 10017-6702, US

(73) Assignee: Genentech, Inc, South San Francisco, CA (US)

(21) Appl. No.: 10/585,881

(22) PCT Filed: Jan. 7, 2005
(86) PCT No.: PCT/US05/00416

§ 371 Date: Jul. 11, 2006

Related U.S. Application Data
(60) Provisional application No. 60/481,891, filed on Jan. 13, 2004.

(51) Int. Cl. G01N 33/53; G01N 33/566

(52) U.S. Cl. 435/7.92; 436/501

(57) ABSTRACT

A single-step immunoassay method, kit, and reagents for detecting and quantifying contaminant host cell proteins in a recombinant protein sample are described. The method includes the step of adding to immobilized medium comprising a capture reagent including anti-host cell protein antibodies, both the recombinant protein sample and a detection reagent comprising anti-host cell protein antibodies and a detectable moeity. The recombinant protein sample and two reagents are added simultaneously. This single-step format provides greater interaction between the capture antibody, the contaminant host cell proteins that may be present in the recombinant protein sample, and the detection antibody. By providing the opportunity for both antibodies and HCPs to interact at the same time, the one step format allows the formation of the “capture antibody-HCP-detection antibody” complex with all possible HCPs present. Thus, the HCP assay sensitivity is significantly improved, and the possibility of a false negative is significantly reduced. In addition, the one step format shortens the assay turnaround time and provides a convenient tool for measuring the HCP in the product during the course of the entire purification procedure. Quality control of the process is increased and the protein purification efficiency during process development can be measured.

### BACKGROUND OF INVENTION

For recombinant biopharmaceutical proteins to be acceptable for administration to human patients, it is important that residual contaminants resulting from the manufacture and purification process are removed from the final biological product. These process contaminants include culture medium proteins, immunoglobulin affinity ligands, viruses, endotoxin, DNA, and host cell proteins. These host cell contaminants include process-specific host cell proteins (HCPs), which are process-related impurities/contaminants in the biologics derived from recombinant DNA technology. U.S. and foreign regulations often require removal of such contaminants. For example, the U.S. Food and Drug Administration (FDA) requires that biopharmaceuticals intended for in vivo human use should be as free as possible of extraneous immunoglobulin and non-immunoglobulin contaminants, and requires tests for detection and quantitation of potential contaminants, such as HCPs. As well, the International Conference on Harmonization (ICH) provides guidelines on test procedures and acceptance criteria for biotechnological/biological products. The guidelines suggest that for HCPs, a sensitive immunoassay capable of detecting a wide range of protein impurities be utilized. Although there are commercial assays and reagents available to detect immunoglobulins, DNA, endotoxins, viruses, etc., there are currently no commercial reagents or analytical methods available for the detection and quantification of process-specific HCPs.

The level of HCP contamination in a given biopharmaceutical will depend on the dosage, duration of use, host cell used to manufacture, etc. For example, an immunoglobulin drug that must be administered in very high doses over long periods of time would require a much lower level of HCP contaminants than a single dose of a vaccine because of the likelihood of adverse immune reactions. Failure to remove these contaminants early in the drug development process can result in reduced efficiency and delay in approval of a given biopharmaceutical drug.

Thus, the development of a sensitive assay and reliable process-specific reagents for detecting and quantifying process-specific HCPs as a whole would improve quality control for development of recombinant biopharmaceutical proteins, as residual HCP content is considered a critical indicator for product quality. Further, residual HCP content is an indicator for purification process efficiency and consistency for recombinant proteins. Thus, developing such an assay and respective reagents would consequently improve efficiency and consistency of the purification process, lower cost of the development process in the long run, and increase the chances that the biopharmaceutical drug will attain approval.

Because there are currently no acceptable, generic assays and associated reagents available for quantitation of process-specific HCPs, the current biopharmaceutical industry consensus methodology for development and validation of process-specific, multi-analyte, HCP immunoassay is based upon the null cell, mock purification model. See, for example, Eaton, L. C., “Host Cell Contaminant Protein Assay Development For Recombinant Biopharmaceutical”, J Chromatogr A 705(1): 105-14 (1995), the disclosure of which is incorporated herein by reference. Under this model, a null cell (a cell similar to or identical to the cell used for production of the biopharmaceutical) is used to generate host cell proteins or other potential contaminants, but is incapable of producing the biopharmaceutical product itself. For example, in a prokaryotic expression system that produces recombinant human growth hormone, the null cell may be a bacterial strain harboring the plasmid vector minus the human growth hormone gene. The host cell proteins from this “null” cell are used to generate polyclonal antibodies from a crude preparation under mock production conditions.

Then, for example, a Western Blot (WB) employing these antibodies can then theoretically detect semi-quantitatively any HCP that is present and that separates on the gel, including those that might co-migrate with the product. The WB can also distinguish between contaminating HCPs and product-related impurities. An enzyme-linked immunosorbent assay (ELISA) can further supplement the WB and provide quantitative data on the total amount of HCP present.

HCPs include a variety of proteins in terms of their molecular weight and biological function. Theoretically, the antibodies for an assay must be polygonal to recognize and detect all possible HCPs. Many small potential HCPs, however, are weak immunogens that do not elicit an adequate antibody response in the host animal. Therefore, an HCP pool may require further modification for the HCPs to be satisfactory multiple immunogens, or else employing special immunization protocols may be necessary. See Hoffman, K., “Strategies for Host Cell Protein Analysis” Biopharm (May 2000), the disclosure of which is incorporated herein by reference. Practically, multi-analyte polyclonal antibodies need to detect as many of the HCPsand only HCPsas reasonably possible. The quality of the antiserum can be monitored with a WB by comparing it with a silver stained gel to show that the majority (i.e. 90%) of the protein bands have corresponding WB bands. The specificity of the antiserum can be monitored with a western blot of known HCP free protein products (i.e., human IgG where the product is a humanized monoclonal antibody) to show that the antiserum does not react with these proteins.

Once a reagent suitable for detection has been developed, an immunoassay sensitive enough to detect contaminant host cell proteins is needed. Some of the proteins to be detected will be in small quantities and/or of a size range not easily detected. Many HCP immunoassays for quantitation of process-specific HCPs have been developed in the biopharmaceutical industry in recent years including: an ELISA for yeast HCPs in recombinant hepatitis B surface antigen (Ohmura, et al., Biochem. Biophys. Res. Comm. 149:1172-1178 (1987)); an ELISA for E. coli HCPs in recombinant human interferon (Chen et al., Appl. Biochem. Biotechnol. 36: 137-152 (1992)); and an ELISA for mouse fibroblast HCPs (Pauly, et al., Behring Inst. Mitt. 86:192-207 (1990)).

SDS-PAGE/Silver Stain offers one method of qualitative analysis of a biopharmaceutical product preparation. It offers good resolution and sensitivity, but suffers from subjective interpretation of band comparison and is technique dependent. HPLC can be quantitative with high resolution, but offers only low sensitivity, as well as being subjective and costly. A western blot offers reasonable sensitivity, but it is only qualitative and the antibodies used may fail to detect some contaminants. Immunoassays offer very high sensitivity and can offer an objective endpoint, but there is no resolution of individual components and again the antibodies used may fail to detect some contaminants. Moreover, typical immunoassays require a two-step process otherwise there can be a significant number of “false negatives” because the assay failed to detect bound antibody.

False negatives result when the HCP detection assay fails to detect an HCP present in the biopharmaceutical product preparation. In other words, some HCPs may not be able to form a complex with the capture and detection antibodies, thus remaining undetectable in the assay, and resulting in a false negative measurement. Thus, there is a need for a sensitive, quantitative assay method, kit, and reagent for the determination of HCP contaminants in a protein product, particularly where prior techniques have failed in terms of consistency, efficiency, and sensitivity.

### SUMMARY OF INVENTION

The present invention provides a single-step immunoassay method for detecting and quantifying contaminant host cell proteins in a recombinant protein sample. The immunoassay can be an ELISA. The method includes a single-step immunoassay method for detecting and quantifying contaminant host cell proteins in a recombinant protein sample wherein a capture reagent comprising anti-host cell protein antibodies, and a detection reagent comprising anti-host cell protein antibodies are added substantially simultaneously to a recombinant protein sample; and the level of host cell protein in the recombinant protein sample is detected and/or quantified.

The capture reagent may be immobilized on a support medium, such as a bead or a microtiter plate. The detection reagent may be (i) purified anti-HCP antibodies from either the same or different animal source labeled with a detectable moiety; (ii) an anti-HCP IgG fraction from a different animal source than that of the capture reagent anti-HCP antibodies; or (iii) undiluted, unfractionated serum containing anti-HCP antibodies from a different animal source than that of the capture reagent anti-HCP antibodies. The detection reagent may include a detectable moiety or may be detected by adding a secondary reagent that recognizes the detection reagent and the secondary reagent has a detectable moeity.

If the capture reagent anti-HCP antibodies and the detection reagent anti-HCP antibodies are from the same species, it may be necessary to affinity purify the antibodies. Thus, in one embodiment, HCPs are attached to a support medium, such as a bead, and used to affinity purify the anti-HCP antibodies generated.

The capture reagent anti-HCP antibodies may be produced by immunizing a first animal species and the detection reagent anti-HCP antibodies may be produced by immunizing a second animal species.

In another embodiment, the immunoassay method for detecting and quantifying contaminant host cell proteins in a recombinant protein sample may be an ELISA assay wherein the capture reagent is immobilized on a support such as a microtiter plate and the recombinant protein sample and a detection reagent comprising anti-host cell protein antibodies are added substantially simultaneously to the plate.

The present invention also includes a reagent for use in a single-step immunoassay method for detecting and quantifying contaminant host cell proteins in a recombinant protein sample, the reagent comprising an affinity purified anti-host cell protein antibody preparation. This reagent may be affinity purified by: preparing an affinity medium comprising host cell proteins coupled to a support and separating anti-host cell antibodies from other compounds using the affinity medium. The capture reagent and the detection reagent may be generated by immunizing two different animal species.

The present invention includes a kit for detecting and quantifying contaminant host cell proteins in a recombinant protein sample as part of an overall assay, the kit comprising (a) a capture reagent comprising an affinity-purified anti-host cell protein antibody preparation; and a detection reagent comprising (i) purified anti-host cell protein antibody and a detectable moeity, or (ii) unpurified anti-HCP IgG fraction from different animal source than that of the capture reagent anti-HCP antibodies, and a detection labeled secondary antibody to this IgG fraction, or (iii) undiluted, unfractionated anti-HCP containing serum from a different animal source than that of the capture reagent anti-HCP antibodies, and a detection labeled secondary antibody to the IgG in the serum. The kit may include a support medium wherein the capture reagent is immobilized. The kit may be an enzyme-linked immunosorbent assay (ELISA).

### BRIEF DESCRIPTION OF DRAWINGS

The accompanying drawings illustrate various embodiments of the present invention and are a part of the specification. The illustrated embodiments are merely examples of the present invention and do not limit the scope of the invention. Throughout the drawings, identical reference numbers designate similar, but not necessarily identical, elements. Together with the following description, the Figures demonstrate and explain the principles of the invention and are views of only particular rather than complete portions of the invention.

FIG. 1 schematically shows the steps of the immunoassay of the present invention.

FIG. 2 shows a graphic summary of titers of different anti-HCP preparations, including crude serum (50), protein A purified anti-HCP, and HCP affinity purified anti-HCP (80).

FIG. 3 shows an example of the use of a standard curve fitting graph to determine HCP concentration.

FIG. 4 shows a graphic comparison of anti-human IgG activity for two lots of crude rabbit serum and HCP affinity purified anti-HCP antibody in ELISA for specificity evaluation.

FIG. 5 shows a graphic comparison of anti-HCP activity for two lots of crude rabbit serum and HCP affinity purified anti-HCP antibody in a sandwich ELISA.

FIG. 6 shows a WB image for evaluation of HCP affinity purified anti-HCP antibody from three lots of rabbit serum.

### DETAILED DESCRIPTION

The following description provides specific details in order to provide a thorough understanding of the invention. The skilled artisan, however, would understand that the invention can be practiced without employing these specific details. Indeed, the present invention can be practiced by modifying the illustrated system and method and can be used in conjunction with apparatuses and techniques conventionally used in the industry. The invention includes a rapid and sensitive quantitative immunoassay for the quantitative measurement of HCP contaminants. Any immunoassay that obtains these functions can be used in the invention. The present invention provides a single-step immunoassay method for detecting and quantifying contaminant host cell proteins in a recombinant protein sample. The single-step assay format of the present invention provides greater interaction between the capture antibody, the HCP, and the detection antibody. The HCP assay of the present invention is performed in a single-step incubation format, where both capture and detection antibodies are incubated simultaneously with the HCPs. By providing the opportunity for both antibodies and HCPs to interact at the same time, the one step format allows the formation of the “capture antibody-HCP-detection antibody” complex with all possible HCPs present. Thus, the HCP assay sensitivity is significantly improved, and the possibility of a false negative is significantly reduced. In addition, the one step format shortens the assay turnaround time and provides a convenient tool for measuring the HCP in the product during the course of the entire purification procedure. Quality control of the process is increased and the protein purification efficiency during process development can be measured.

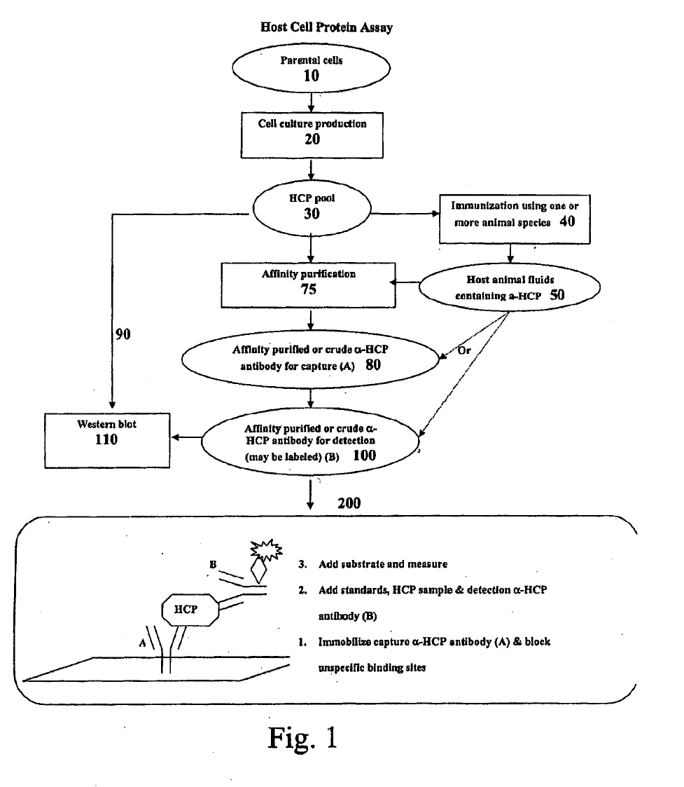

In one aspect of the invention, the immunoassay of the present invention is outlined in the flowchart of FIG. 1. All numbers appearing in the text refer to steps in the flowchart of FIG. 1.

As shown in FIG. 1, parental cells (10) of a single line of cells are obtained and prepared for culturing to produce HCPs. The cells (10) are subjected to the same cell culture production process (20) as the recombinant protein product, i.e. a “mock” production. This process should mimic the growth conditions, medium, etc. that the recombinant protein product undergoes in order to obtain the same HCPs.

The HCP pool (30) is obtained from this mock production by (1) the supernatant from the cell culture, (2) by homogenizing the cells and collecting the resulting supernatant, or (3) by whatever process that is likely to yield all possible HCPs. This crude HCP pool (30) contains all possible HCPs that one would encounter during recombinant protein production. The HCP pool may be concentrated and stored at −80° C.

The HCP preparation may be analyzed to: (1) quantitate the amount of protein present in the sample, (2) characterize the HCPs present, and (3) qualitate the HCPs to verify that no recombinant protein product is present in this mock production.

Commonly used detection techniques for quantifying proteins include isotopic, calorimetric, fluorometric luminescent or immunoassay-based procedures with isotopic or color endpoints. The quantitation may be done by any of these methods. One such assay is a bicinchoninic acid (BCA) assay.

Characterization of proteins is commonly done via a SDS-PAGE/silver stain method, allowing a qualitative determination of the proteins present based on molecular weight. A Western Blot (WB) probed with labeled anti-product antibodies may be used as an assurance control to ensure that the preparation is free of any recombinant protein product, as well as a verification of the silver-stain assay. This characterized HCP pool (30) may then be used for: (1) animal immunization (40), (2) as a coupling immunogen to make an affinity medium (75), and (3) as a standard in the ELISA assay (200). This immunogen source is one component of the immunoassay process of the present invention.

The HCP pool (30) may be used as an immunogen source in the generation of polyclonal antisera. In one aspect of the invention, a host animal, such as rabbit, rodent, guinea pig, etc., may be used for the immunization treatment (40). See e.g., Colco, Current Protocols in Immunology, John Wiley and Sons, Inc. 1995. The proteins and/or peptides can be suspended or diluted in an appropriate physiological carrier for immunization. Suitable carriers are any biologically compatible, non-toxic substance to deliver and/or enhance the immunogenicity of the peptides, including sterile water and 0.9% saline. Alternatively, the peptides can be coupled to a carrier molecule before being used as an immunogen.

Immunogenic amounts of antigenic preparations enriched for the desired epitopes are injected, generally at concentrations in the range of 1 μg to 20 mg/kg body weight of host. Administration can be by injection, e.g., intramuscularly, peritoneally, subcutaneously, intravenously, etc. Administration can be one time or a plurality of times, usually at one to four week intervals. Immunized animals are monitored for production of antibody. An exemplary immunization treatment is based on a seven-day routine. Animals are pre-bled on day 0. An initial injection (e.g., subcutaneous, 1 mg each) is administered in complete Freund's adjuvant (CFA), and all subsequent injections (every two weeks, 1 mg each) are in incomplete Freund's adjuvant (IFA). Animal fluid, such as sera (50), is collected from each animal seven days after each boost. The titer from the first bleed may be assessed using, e.g., an enzyme immunoassay (EIA) or an enzyme linked immunosorbent assay (ELISA). All bleeds including the pre-bleed are analyzed for HCP-specific and non-specific activities by, e.g., WB.

The crude polyclonal anti-HCP antibodies (50) are collected and, under certain conditions, may require purification. The antibodies may be purified using, e.g., an HCP affinity chromatography column (75). Such affinity purification methods generally utilize insoluble or immobile protein conjugates to facilitate separation of the antibody from host proteins, etc. Purification methods are described by Harlow et al. (1988) Antibodies: a laboratory manual. Cold Spring Harbor Laboratory, Cold Spring Harbor, N.Y. Appropriate matrices may include but are not limited to agarose, latex, magnetic or polyacrylamide beads, silica or polystyrene. Other purification methods, such as ion exchange chromatography, gel filtration chromatography, or preparative gel electrophoresis, may also be used to increase the purity of the preparation.

The affinity media can be made, for example, by covalently coupling HCPs from the initial HCP pool (30) to support beads or matrix material, such as CNBr-activated beads. The HCP coupled affinity beads may be packed into a column and the crude diluted serum (50) loaded onto the column. Typically, the column is washed, eluted, and then followed by an acid strip. The loading flowthrough may be collected and passed through the column again to capture any anti-HCP antibodies that did not bind the first time through. The elution, acid strip, and the flowthrough fractions are evaluated using EIA and WB for titers and specificity. Alternatively, the HCP-coupled beads may be used to purify the anti-HCP antibodies by a batch affinity method.

These affinity purified anti-HCP antibodies (80) may be conjugated to a detection label, such as horseradish peroxidase (HRP), for the detection of HCPs present in the biopharmaceutical product. The conjugated antibody (100) is separated from any unconjugated antibody. A conjugate stabilizer may be added, such as bovine serum albumin (BSA), sucrose and glycerin. This labeled anti-HCP antibody is used for detection in the HCP assay ELISA format (200) to be discussed below.

Another component of the immunoassay process of the present invention is the affinity purified anti-HCP antibody. This anti-HCP antibody is used both as the primary antibody in the WB analysis and as the capture (coating) antibody in the ELISA analysis (200). Since both the capture and detection antibodies are polyclonal and from the same source, it can be assumed that they recognize different epitopes on the same HCP. Other examples include using polyclonal anti-HCP antibodies from different animal species. Antibody from one animal species can be used as the capture antibody and antibody from another species used as the detection antibody and probed by a secondary antibody specifically against the antibody from the second animal species.

The assay format of the present invention is a single-step sandwich assay including a single antibody incubation step and a single enzyme reaction step. The total assay time is about 4 hours. The assay is sensitive, with the limit of detection (LOD) at nanogram levels and the limit of quantitation (LOQ) is at 16 ng/ml, while being specific and robust. It reduces the possibility of a false negative response. Consequently, the assay enhances the ability to monitor efficiency and consistency in recombinant biopharmaceutical protein product purification processes.

The invention is described generally above, but is described in more detail below. In the description below, a specific portion of the method illustrated in FIG. 1 is generally described. An example is presented for each described portion of the method.

### EXAMPLES

### Example 1

### HCP Pool (30) Production from Parental Cells (10)

Frozen parental cells were thawed, in a 37° C. water bath for an appropriate amount of time. The thawed cells were mixed, and an aliquot (i.e. 1 ml) of the cells taken and mixed with a pre-warmed growth medium (at about 37° C.). The ratio of the growth medium to the cell concentration was about 9:1. The cell culture was then centrifuged to form a cell pellet to better separate the cells from impurities. The cell pellet was re-suspended in a similar amount of growth medium. The cell culture was incubated at about 37° C. (i.e., about 90% humidity and about 5% CO₂). The cell culture was transferred to a spinner flask, and after about 7 days to a bioreactor. Once the cells were ready to harvest, the cell culture was centrifuged and filtered using a 0.2 μm filter.

A BCA protein assay was performed to determine the total protein concentration. A well known BCA technique and kit uses a combination of working reagents, which are then mixed with amounts of individual standard or unknown protein samples in sample tubes. The samples or controls are incubated (i.e., for about 30 minutes) and then allowed to cool to room temperature before measuring their individual absorbencies (i.e., at 562 nm against a water reference). Each sample's protein concentration was calculated based on a standard curve with linear regression.

Small molecules, such as some amino acids in the cell culture medium, have the potential to interfere with the BCA assay. Therefore, such small molecules should be removed from the protein solution before performing the BCA assay in order to get accurate results for protein concentration. To separate the proteins from the small molecules, a stirred cell ultrafiltration or tangential flow filtration using an ultrafiltration (UF) membrane with a molecular weight cutoff of 3,000-5,000 daltons can be used.

10 L of cell culture supernatant (Protein conc.—0.3 mg/ml) from an HCP production was divided into 1 L aliquots. 2 L of the medium was concentrated and buffer exchanged using a stirred cell with YM3 membranes to a final volume 180 ml at 2.72 mg/ml. This result was achieved by the following process. Five YM3-concentrated HCP batches were combined and dialyzed against phosphate buffered saline (PBS), pH7 through a dialysis membrane with a 6,000-8,000 MW cutoff (flat width 100 mm, Diameter 64 mm). Dialysis tubing (20 cm) was soaked in Milli-Q water followed by PBS, each for 10 minutes prior to use. A total of 250 ml of HCP concentrate was dialyzed against 2L of PBS at 2-8° C. for 36 hours with four changes of buffer approximately every 6-10 hours. About 200 ml of HCP in PBS solution was removed from the dialysis tubing and sterile filtered through 0.2 μm syringe filters. The yield was about 80%. This lot of HCP preparation was designated “HCP pool I.” The HCP pool I was analyzed via a BCA assay for HCP concentration, via silver stained SDS-PAGE, and WB probed with anti-human antibodies for banding patterns as a quality control.

6 L of the medium was used for diafiltration via ultrasette tangential flow filtration cassette with a 5,000 MW cutoff membrane. The final concentration was 3.3 mg/ml for a total of 500 ml. This result was achieved by the following process. The 6 L of cell culture supernatant was concentrated in six batches to a final volume of 1 L, with the membrane regenerated by flushing PBS between the batches. A buffer exchange to PBS (pH7) was performed via diafiltration using the same ultrasette. 2 L of PBS was slowly siphoned into the feed/retentate bottle that contained 1 L of concentrated HCP cell culture supernatant, and the HCP was concentrated to a final volume of about 500 ml. This buffer exchange procedure was done twice with a total buffer exchange efficiency of about 94%.

50 mg of thimerosal was added to 500 ml of HCP concentrate in a final concentration of 0.01% to prevent bacteria growth. This preparation was designated “HCP pool II.” 15 ml aliquots of HCP pool II were stored in 15 ml centrifuge tubes at 70° C. The concentration was determined using BCA assay. SDS-PAGE, Western Blot, and analytical size exclusion chromatography (SEC) were performed for characterization of the proteins in the HCP pool (30).

HCPs from a known recombinant protein production was used for comparison. These HCPs were obtained from the cell culture supernatant, which was further separated from the biopharmaceutical product via an immobilized protein A affinity chromatography (IPA). The unbound IPA fraction (flowthrough), which contains the majority of the HCPs, was collected and concentrated via ultrafiltration.

The protein concentration of the HCP pool I (after dialysis and sterile filtration) was 2.72 mg/ml. The HCP pool II (after diafiltration) had a protein concentration of 3.30 mg/ml. The SDS-PAGE profiles for the concentrated HCP pool I and II were similar to the crude HCP production cell culture supernatant. When compared to the biopharmaceutical product reference standard, the HCP pools are absent of the antibody heavy and light chains bands, which indication no product contamination. When compared to the IPA unbound fraction, the HCP pools showed majority of the bands found in the IPA unbound fraction. This proofs that the HCP pools are representative of the HCP from the real biopharmaceutical production.

The results of the HCP characterization were supported by the results of a WB analysis. This analysis confirmed that there was no contamination of the humanized or chimeric IgG products (such as the recombinant protein used as a reference standard) in the HCP preparation. In this analysis, 0.5 μg protein samples were loaded into a 15 well gel in three sections with each section containing one set of sample along with a pre-stained MW marker. An electroblotting was then performed. The SDS gel was blotted to 0.45 μm nitrocellulose membrane in a Tris-Gly transfer buffer containing 20% methanol. The transfer was done at 80 V for 1 hour in an ice-cold buffer tank. After transfer, the membrane blot was soaked in Blotto (5% dry milk in PBS buffer containing 0.1% TWEEN™ 20) overnight at 2-8° C. After 3 PBST (PBS with 0.1% TWEEN™ 20) washes, the membrane was cut into three strips with each strip containing a set of a sample and a pre-stained MW marker. The individual strip was probed with goat anti-human antibody HRP conjugates. The blot was then incubated with a 1:5000 dilution of antibody conjugates in 5 ml of Blotto for 2 hours at 15-30° C. with gentle shaking. After the blot was washed three times with PBST, it was developed with a TMB/peroxidase substrate for several minutes until the color developed. The blot was then rinsed with water and the image was scanned using an imaging densitometer.

On the blot, the humanized antibody reference standard (which reacts with all the anti-human antibodies) was compared with the HCP pool I and II samples, and with the IPA flowthrough sample. The HCP pools were negative to all anti-hu IgG antibodies. The IPA flowthrough sample revealed the presence of antibody light chain.

The size exclusion chromatography (SEC) profiles of the concentrated HCP preparations were similar to that of the crude HCP production cell culture supernatant. There were three major peaks at about 10, 15 and 19 minutes, representing MW ranges at greater than 670K, about 300K, and 50K Daltons. The first two peaks were dominant with peak area ranges from 30% to 40%. The IPA flowthrough sample results revealed the same number of peaks but with different intensity. The second peak was dominant with about 50% peak area while the first one was the least intensive with only 5% peak area.

The total HCP preparation yielded about 2 grams of HCP. It was determined to be free of antibody contaminants, and represented the host cell protein population in the real antibody production using the same cell line and process. All HCP pools were stored in 10-15 ml aliquots at −70° C. for subsequent use in rabbit immunization, HCP affinity column preparation (75), and as an ELISA reference standard.

### Example 2

### Immunization (40)

The HCP pools (30) are then utilized for custom polyclonal antisera production. An example immunization treatment as discussed above and as produced in Example 2 is based on a seven-day routine.

The two HCP preparations, pools I and II in 10 ml aliquots, were subjected to custom polyclonal antisera production procedures. Two New Zealand White rabbits for each immunogen were used with a standard immunization protocol based on a seven-day routine. Animals were pre-bled on day 0. The initial injection (1 mg each) was administered subcutaneously in complete Freund's adjuvant (CFA) and all subsequent injections (every two weeks, 1 mg each) were in incomplete Freund's adjuvant (IFA). About 25 ml of sera were collected from each rabbit seven days after each boost. The titer from the first bleed was assessed using EIA. All of the rabbits exhibited good binding titers to the HCP immunogens (titer is defined as the antiserum dilution that produces a net OD of 1.0 in the EIA assay).

All bleeds including the pre-bleed were analyzed for HCP specific and non-specific activities by WB. The purpose of the WB is two-fold. First, the WB allows the banding pattern of the HCP pool I and II antisera to be compared with silver stained SDS gel to assess the quality of the immunization previously performed. Second, the WB provides an assessment of the specificity of the antisera, including any pre-existing anti-human IgG activity. The WB procedure used is similar to the one described in Example 1, with some variations. The reduced SDS-PAGE gels were run according to the standard procedure. The HCP pools and the recombinant protein reference standard were loaded to the gel (0.1 μg and 1.0 μg/well for HCP sample, 10 μg/well for reference standard). The gels were transferred to a nitrocellulose membrane in a transfer buffer at 20V for 20 min/gel. The blots were blocked in Blotto overnight at 2-8° C. After 3 washes with PBST, the blots were incubated with a 1:500 dilution of affinity purified anti-HCP antibody (80) in Blotto for 12 hours with gentle shaking. After another 3 washes with PBST, the blots were incubated with goat anti-rabbit IgG (H+L) HRP conjugate for 1 hr with shaking. After being washed 3 times (5 min each) with PBST, the blots were developed with TMB/peroxidase substrate, and then scanned using an imaging densitometer.

### Example 3

### HCP Affinity Chromatography (75)

As mentioned above, the crude polyclonal anti-HCP antibodies produced from the sera are purified using HCP affinity chromatography. The affinity media includes HCPs from the initial HCP pool (30) that were coupled to beads. The HCP-coupled affinity beads were packed into a column. Crude diluted rabbit serum (50) was loaded onto the column. The buffered serum was washed, eluted, and then followed by an acid strip. The loading flowthrough was also collected and subjected to recapture for any unbound HCPs. The elution, acid strip, and the flowthrough fractions were evaluated using EIA and WB for titers and specificity.

Five grams of CNBr activated Sepharose 4 Fast Flow beads (Amersham Biosciences, Uppsula, SE) was hydrated in PBS, pH 7 buffer in a 50 ml centrifuge tube, yielding about 20 ml of drained gel. 12 ml of concentrated HCP preparation in PBS (5.14 mg/ml) via stirred cell with YM3 membrane was added to the drained gel to form a slurry. The slurry was incubated at 15-30° C. for 4 hours with gentle shaking followed by incubation at 2-8° C. overnight. The progress of the coupling of the beads and the HCPs was monitored by taking the OD₂₈₀ of 50 μl aliquots of the clear supernatant (Diluted 20× in PBS) at time 0 hours, 1 hour, 2 hours, and 20 hours of the incubation. After 20 hours, the coupling reaction was stopped by decanting the clear supernatant and changing to TBS buffer, pH 8.5 (50 mM Tris, 27 mM NaCl). The coupling yield was about 84% after 2 hrs, and 87% after 20 hrs, yielding about 15 ml of drained affinity resin.

The HCP-coupled beads were washed alternately with TBS, then sodium acetate buffer (50 mM sodium acetate, pH 4.36) three times by centrifugation, decanting the clear supernatant, and adding the fresh buffer. After two wash cycles, about 15 ml of drained gel remained. The beads were stored in PBS, pH 7 with 20% ethanol at 2-8° C. until use in the affinity column.

The HCP-coupled beads were then packed to a Bio-Rad Econo column (i.d.=1.5 cm, bed height=9 cm, bed volume=15 ml) in PBS containing 1 M NaCl (pH 7) using gravity. The column was flushed and equilibrated with 10 column volume (CV) of PBS, pH 7.4 at 5 ml/min. Up to 25 ml of crude rabbit serum diluted with an equal volume of PBS was loaded onto the column. The loaded column was washed with 10 CV of PBS, eluted with 5 CV of glycine pH 3.0, and then followed by an acid strip with 0.1N HCl. The loading flowthrough was collected and subject to affinity purification one more time. The glycine eluted fraction, acid strip, and the flowthrough fractions were evaluated using EIA and WB for titers and specificity.

### Comparative Example 3A

### Protein A Purification of Anti-HCP Antisera

To comparatively determine the effectiveness of the anti-HCP affinity column, a protein A purification of the anti-HCP antisera was performed.

1 ml of Rabbit anti-HCP serum was diluted with 1 ml of PBS, pH 7.4 and loaded onto a ProSep A column (i.d.=1.5 cm, bed L=14 cm, vol=25 ml) at 5 ml/min. After 10 CV of wash with PBS (pH 7.4), the antibody was eluted with 0.2 M glycine (pH 3.0) followed by an acid strip step with 0.1 M HCl. The glycine eluted fraction, acid strip, and the flowthrough fractions were evaluated for titer using EIA.

According to the EIA process, a plate (Immulon 2 HB) was coated with either HCP pool I or II. 50 μl of different anti-HCP antibody dilutions in a reference matrix was added to the plate. Immediately, 150 μl of a 1:25,000 dilution of goat anti-rabbit IgG (H+L) HRP conjugate in an assay buffer was added to the wells. The plate was incubated for 1 hour with shaking at 15-30° C. Following the incubation, the plate was washed with wash buffer. 100 μL of TMB substrate was then added and allowed to incubate for 10 min with shaking. After 10 min, 50 μl of stop solution was added. The absorbance was measured at 450 nm with a 630 nm reference filter.

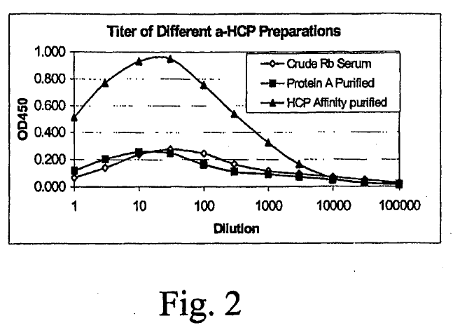

FIG. 2 shows the results of the titers for each of the anti-HCP antibody preparations. The binding activity of the protein A purified antibody (95) to HCP showed no significant improvement over the crude antiserum, while the HCP-affinity purified antibody had a 4-fold increase in binding activity over that of the crude. Thus, the HCP-affinity column enriched the antibody in a remarkable and efficient manner.

### Example 4

### Western Blot (110)

A WB was performed in order to evaluate the selectivity of the column and the specificity of the affinity purified anti-HCP antibody (80). A recombinant protein reference standard, an crude HCP production cell culture supernatant, and the two HCP pools I and II were individually loaded onto a 15-well gel and transferred to a membrane in three panels. These three panels were probed with either the crude antiserum, the affinity column flowthrough, or the affinity purified anti-HCP antibody (80). Both the crude antiserum and the affinity flowthrough showed a positive band to the heavy chain of the reference standard, while the affinity purified antibody was negative to the reference standard. The flowthrough fraction did not react to the HCP preparations significantly, while both the crude and the purified did. The WB results show that the HCP-affinity column enriches the antibody selectively, and the purified anti-HCP antibody is specific to the HCPs, and not antibody products.

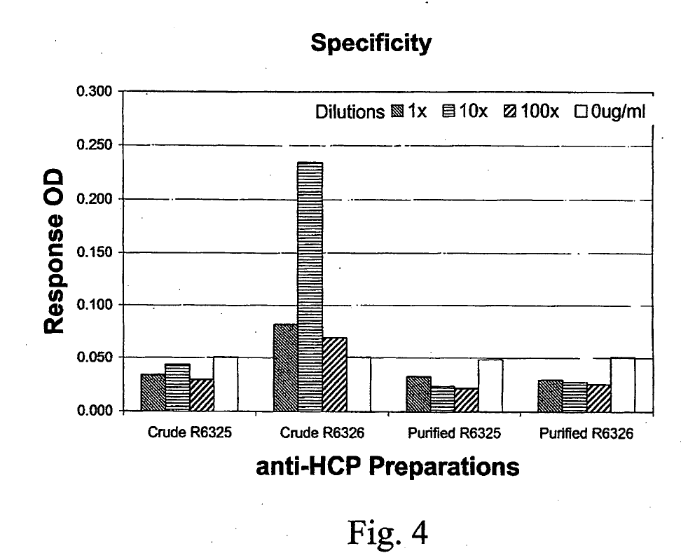

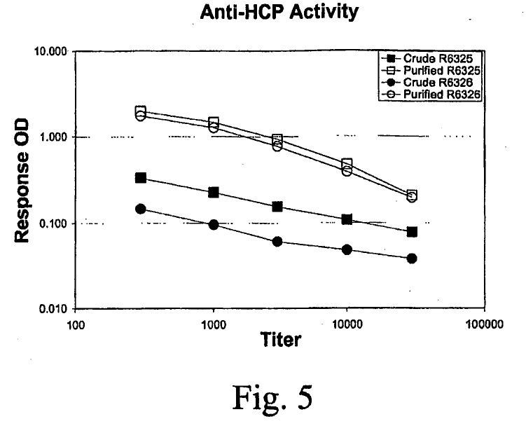

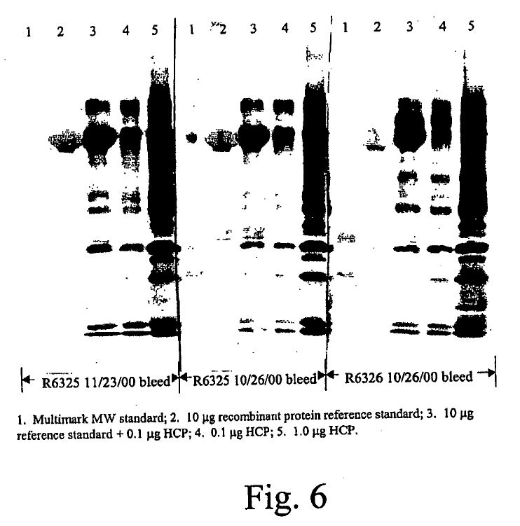

To supplement the above-discussed small-scale purification and related assays, multiple runs of slightly larger scale (25 ml serum per run) were performed using the same HCP-affinity column. The serum and its corresponding purified antibody were labeled according to the bleed dates during immunization. A total of 150 ml of serum was purified in 6 runs. The flowthrough was collected, combined for every two runs, and reloaded for the second time. The antibody titer and specificity were monitored using EIA, WB, and/or ELISA with human-immunoglobulin G (hu-IgG) coated plates. The results showed there was a 5-10 fold increase in titers over the crude sera. These antibodies react specifically to HCP and not to hu-IgG. The second pass affinity purified antibodies showed comparable activity and specificity. Typical ELISA- and WB results are shown in FIGS. 4 to 6.

### Example 5

### Anti-HCP Antibody HRP Conjugate Preparation (100)

Affinity purified anti-HCP antibodies obtained by the above methods were conjugated to HRP. The conjugated antibodies were separated from the unbound antibodies using a desalting column, and then concentrated to a final volume. A conjugate stabilizer was added to this concentrated solution.

Affinity purified anti-HCP antibodies (80) were conjugated to HRP by the following procedure. 8 ml of affinity purified anti-HCP antibodies (0.407 mg/ml) were concentrated and buffer exchanged to 0.1 M sodium bicarbonate buffer (pH 9.5) using Bio-Rad BioMax-30K 15 ml concentration filters, to a final concentration of 10 mg/ml. Prior to conjugation, one vial of the activated enzyme was dissolved with 400 μl of 0.1 M sodium bicarbonate buffer. To 250 μl of this enzyme solution, 50 μl of the concentrated antibody was added. The mixture was incubated at 15-30° C. for 2 hours with gentle shaking. To block the remaining active sites, 100 μl of 1M lysine solution was added to the reaction mixture and the mixture was incubated for one hour at room temperature. Finally, the conjugated antibodies were separated from the unbound antibodies using a Biorad EconoPac P6 desalting column followed by concentration using Centricon YM30 to a final volume of 100 μl. 200 μl of a conjugate stabilizer was added. The resulting conjugate (100) was used in the ELISA assay as detection antibody.

### Example 6

### ELISA Assay (200)

The purified anti-HCP antibody was used as the capture antibody in the ELISA analysis and the HRP-labeled anti-HCP antibody was used as the detection antibody. Since both the capture and detection antibodies are polyclonal, they must recognize different epitopes on the same HCP. Alternatively, the detection labeled anti-HCP conjugate can be replaced by unpurified anti-HCP IgG fraction or anti-HCP serum from a different species of animal (Second anti-HCP), such as goat. After incubation of anti-HCP with HCP, a secondary antibody with a detection label is added to this second anti-HCP.

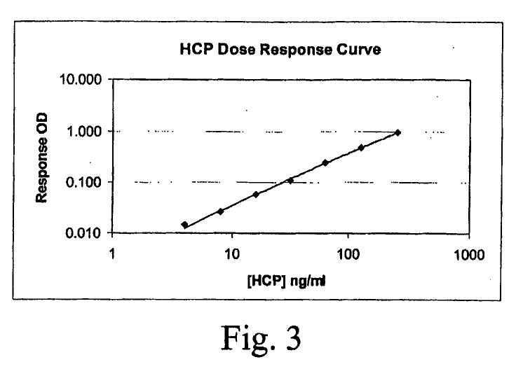

In this example, a plate (Immulon HB II) was coated with 7.5 μg/ml of affinity purified anti-HCP antibody (80) in PBS (pH 7.4) and blocked with 1% BSA in PBS. 50 μl of reference standard, or inter-assay controls, or test samples, diluted in reference matrix (50 mM Tris, 0.9% NaCl, 5% BSA and 0.05% ProClin-300) in replicates, were added to the coated plate. Immediately, 100 μl of anti-HCP HRP conjugate (1:3000 dilution in assay buffer: 50 mM Tris, 0.9% NaCl, 0.5% BSA, 0.05% ProClin-300 and 5% normal rabbit serum) was added to each well. The plate was incubated at 15-30° C. for 1-3 hours with fast shaking (700 rpm). After being washed with TBS, the plate was incubated with 100 μl of TMB/peroxidase substrate for 10 min with shaking. After 10 min, 50 μl stop solution was added to quench the color development. The absorbance at 450 nm was measured with 630 nm as reference filter. The sample HCP concentration was calculated based on the standard curve with a quadratic curve fitting. FIG. 3 shows an example of the use of a standard curve fitting graph to determine HCP concentration. The assay system suitability and precision is controlled by evaluating the standard curve and the positive spiking control as described below.

### ELISA Assay Qualification

Assay dynamic range: 8-256 ng/ml, 6 points were used in the standard curve quadratic fitting y=ax²+bx+c. Intra-assay precision: One assay was performed containing 16 replicates of each control. The mean, standard deviation, relative standard deviation (RSD), and average recovery (AR) were obtained for each control. The RSDs for the controls containing HCP levels at 8, 32 and 256 ng/ml are 16.15%, 9.48% and 8.72%. The AR is between 97-107%. The data is shown in Table 1.

TABLE 1
Intra-Assay Precision

| | LEVEL | | |
|---|---|---|---|
| | LOW | MID | HIGH |
| [HCP] ng/ml | 8 | 32 | 256 |
| Sample # | | | |
| 1 | 9.48 | 35.09 | 224.15 |
| 2 | 7.14 | 32.20 | 223.88 |
| 3 | 4.50 | 31.04 | 198.47 |
| 4 | 6.26 | 25.52 | 238.47 |
| 5 | 7.43 | 35.09 | 260.84 |
| 6 | 9.19 | 34.22 | 267.64 |
| 7 | 8.31 | 33.36 | 257.17 |
| 8 | 8.31 | 33.07 | 255.34 |
| 9 | 9.48 | 32.49 | 249.55 |
| 10 | 8.60 | 27.85 | 278.33 |
| 11 | 8.31 | 34.51 | 265.55 |
| 12 | 8.01 | 36.54 | 243.49 |
| 13 | 9.19 | 35.09 | 247.18 |
| 14 | 7.43 | 29.30 | 222.29 |
| 15 | 7.72 | 30.17 | 250.87 |
| 16 | 7.72 | 29.59 | 220.69 |
| n | 16 | 16 | 16 |
| MEAN | 7.94 | 32.20 | 244.00 |
| SD | 1.28 | 3.05 | 21.28 |
| RSD % | 16.15 | 9.48 | 8.72 |
| AR % | 99.26 | 100.61 | 95.31 |

To determine the reproducibility of the assay, the assay with a standard curve and multiple replicates of the three controls was performed at least one assay everyday for 11 days. The control concentration was averaged and the RSD was calculated. The RSDs are below 10% and average recovery is between 99-108%. The results are shown in Table 2.

TABLE 2
Inter-Assay Precision

| | LEVEL | | |
|---|---|---|---|
| | LOW | MID | HIGH |
| [HCP] ng/ml | 8 | 32 | 256 |
| SAMPLE # | | | |
| 1 | 9.42 | 35.40 | 271.30 |
| 2 | 8.11 | 31.69 | 252.10 |
| 3 | 7.77 | 32.30 | 257.80 |
| 4 | 8.57 | 37.50 | 281.98 |
| 5 | 8.80 | 29.44 | 261.39 |
| 6 | 7.52 | 32.74 | 248.31 |
| 7 | 9.41 | 35.46 | 210.04 |
| 8 | 7.92 | 32.71 | 269.78 |
| 9 | 8.94 | 34.22 | 253.43 |
| 10 | 9.95 | 35.04 | 213.14 |
| 11 | 8.47 | 31.35 | 277.85 |
| n | 11 | 11 | 11 |
| MEAN | 8.63 | 33.44 | 254.28 |
| SD | 0.77 | 2.31 | 23.68 |
| RSD % | 8.89 | 6.91 | 9.31 |
| AR % | 107.82 | 104.50 | 99.33 |

Sensitivity (Limit of Detection, LOD): The sensitivity of the assay was calculated using the standard deviation and that of the lowest reference standard at 4 ng/mL (Data is shown in Table 3). Using the formula according to the ICH guideline in analytical method validation, the limit of detection: LOD=3.3 σ/S=3.3×0.008/0.005=5.3 ng/ml. Where σ=standard deviation of the lowest response at 4 ng/mL S=standard deviation of the blank.

TABLE 3
Sensitivity

| | [HCP] | [HCP] |
|---|---|---|
| | (ng/ml) | (ng/ml) |
| Plate # | 0 | 4 |
| 51701A | 0.031 | 0.041 |
| | 0.024 | 0.037 |
| | 0.027 | 0.044 |
| | 0.032 | 0.050 |
| 51701B | 0.016 | 0.025 |
| | 0.017 | 0.022 |
| | 0.017 | 0.030 |
| | 0.020 | 0.028 |
| 51601A | 0.023 | 0.039 |
| | 0.026 | 0.039 |
| | 0.026 | 0.033 |
| | 0.024 | 0.042 |
| 51601B | 0.021 | 0.033 |
| | 0.022 | 0.032 |
| | 0.023 | |
| | 0.021 | |
| n | 16 | 14 |
| MEAN | 0.02 | 0.04 |
| SD | 0.005 | 0.008 |

Lower Limit of Quantification (LLOQ): According to ICH guidelines, the Lower Limit of quantification was LLOQ=10 σ/S=10×0.008/0.005=16 ng/ml. However, the average recovery of the lowest point on the quadratic fitted standard curve is the limiting factor. With 8 ng/ml giving satisfactory recovery between 80-120%, it is therefore set as the lower limit.

Spike-recovery: 20 μl of known HCP at two levels (low and middle) were spiked to 980 μl of recombinant protein reference standard at two concentrations (0.2, 1.0 mg/ml). The recovery ranged from 104% to 118% with the highest average recovery 112.4%. Thus, there is little interaction between the recombinant protein of interest and its host cell proteins. It also suggests that the matrix effect, if there is any, are negligible.

Linearity (Linearity of dilution): HCP samples come with a wide range of concentrations. They must be diluted within the assay range. Therefore, the linearity is used to ensure that the results are reliable after dilution. The test results for two unknown samples at 4 different dilutions are listed in Table 4. The RSD for both tests was below 10%. This demonstrated a linear relationship for the dilution within the assay range.

TABLE 4
Linearity

| Dilution | [HCP] | Sample [HCP] |
|---|---|---|
| Factor | Dilution | (ng/ml) |
| 1 | 76.38 | 1527.69 |
| 2 | 43.64 | 1745.71 |
| 4 | 23.31 | 1865.06 |
| 8 | 11.48 | 1836.10 |
| | Average | 1743.64 |
| | SD | 152.68 |
| | RSD % | 8.76 |
| 1 | 32.87 | 657.48 |
| 2 | 15.23 | 609.11 |
| 4 | 7.47 | 597.53 |
| 8 | 3.85 | 615.29 |
| | Average | 619.85 |
| | SD | 26.14 |
| | RSD % | 4.22 |

Specificity: When the HCP assay is performed for human antibodies IgG1, IgG4, and whole IgG from sources other than cell culture (such as human myeloma plasma, and plasma), the result is negative. When the assay is performed for recombinant humanized or chimeric antibodies generated from the cell culture using the similar process, the result is positive. Thus, the HCP assay is specific to the endogenous HCPs.

The above description and examples reveal a rapid HCP immunoassay developed for recombinant protein products generated from a particular parentral cell line. The assay is sensitive (with detection limit at sub ng/ml and the quantitation limit at ng/ml levels), specific, and robust. The development of the HCP assay not only improves product quality control, but also enhances process development the ability to monitor purification process efficiency and consistency for all recombinant proteins. The same development principle is applicable to the development of similar assays for different cell lines as well.

The preceding description has been presented only to illustrate and describe embodiments of the invention. It is not intended to be exhaustive or to limit the invention to any precise form disclosed. Many modifications and variations are possible in light of the above teaching. For example, although the method as outlined above describes in very particular detail the process for detecting and quantifying HCPs generically, the method as described would be readily modified as necessary to suit any particular immunoassay to detect and quantify HCPs in any given product of recombinant protein or proteins.

1. A single-step immunoassay method for detecting and quantifying contaminant host cell proteins in a recombinant protein sample, the method comprising:
a) adding to a recombinant protein sample
i) a capture reagent comprising anti-host cell protein antibodies, and
ii) a detection reagent comprising anti-host cell protein antibodies;
wherein the capture reagent and the detection reagent are added substantially simultaneously; and
b) detecting the level of host cell protein in the recombinant protein sample.

2. The method of claim 1, wherein the capture reagent is immobilized.

3. The method of claim 2, wherein the capture reagent is immobilized on a bead.

4. The method of claim 1, wherein the detection reagent further comprises a detectable moiety.

5. The method of claim 1, wherein the detection reagent is detected by adding a secondary antibody that recognizes the detection reagent and wherein said secondary antibody comprises a detectable moeity.

6. The method of claim 1, wherein the anti-host cell protein antibodies are affinity purified.

7. The method of claim 6, wherein the anti-host cell protein antibodies are affinity purified by:
a) preparing an affinity medium comprising host cell protein coupled to a support; and
b) separating anti-host cell antibodies from other compounds using said affinity medium.

8. The method of claim 1, wherein the detection reagent comprises:
a) purified anti-HCP antibodies from either the same or different animal source labeled with a detectable moiety; or
b) an anti-HCP IgG fraction from a different animal source than that of the capture reagent anti-HCP antibodies; or
c) undiluted, unfractionated serum containing anti-HCP antibodies from a different animal source than that of the capture reagent anti-HCP antibodies.

9. The method of claim 1, wherein the anti-HCP antibodies are produced by immunizing an animal with host cell proteins, and wherein the capture reagent anti-HCP antibodies are produced by immunizing a first animal species and the detection reagent anti-HCP antibodies are produced by immunizing a second animal species.

10. An immunoassay method for detecting and quantifying contaminant host cell proteins in a recombinant protein sample, the method comprising:
a) immobilizing a capture reagent comprising anti-host cell protein antibodies; and
b) adding to said immobilized capture reagent a recombinant protein sample and a detection reagent comprising anti-host cell protein antibodies, wherein the detection reagent and the recombinant protein sample are added substantially simultaneously.

11. The method of claim 10, wherein the immunoassay is an enzyme-linked immunosorbent assay (ELISA).

12. The method of claim 10, wherein the anti-host cell protein antibody preparation is affinity purified.

13. A reagent for use in a single-step immunoassay method for detecting and quantifying contaminant host cell proteins in a recombinant protein sample, the reagent comprising an affinity purified anti-host cell protein antibody preparation.

14. The reagent of claim 13, wherein the antibody preparation is affinity purified by: preparing an affinity medium comprising host cell proteins coupled to a support; and separating anti-host cell antibodies from other compounds using said affinity medium.

15. The reagent of claim 14, wherein the anti-HCP antibodies are produced by immunizing an animal with host cell proteins, and wherein the reagent anti-HCP antibodies are produced by immunizing two different animal species.

16. The reagent of claim 13, wherein the affinity purified anti-host cell protein antibody preparation is a capture reagent and a detection reagent in an enzyme-linked immunosorbent assay (ELISA).

17. A kit for detecting and quantifying contaminant host cell proteins in a recombinant protein sample as part of an overall assay, the kit comprising:
a) a capture reagent comprising an affinity-purified anti-host cell protein antibody preparation; and
b) a detection reagent comprising:
i) purified anti-host cell protein antibody and a detectable moeity, or
ii) a detection reagent comprising unpurified anti-HCP IgG fraction from different animal source than that of the capture reagent anti-HCP antibodies, and a detection labeled secondary antibody to this IgG fraction, or
iii) a detection reagent comprising undiluted, unfractionated anti-HCP containing serum from a different animal source than that of the capture reagent anti-HCP antibodies, and a detection labeled secondary antibody to the IgG in the serum.

18. The kit of claim 17, wherein the antibody preparation is affinity purified.

19. The kit of claim 17, wherein the anti-HCP antibodies are produced by immunizing an animal with host cell proteins, and wherein the reagent anti-HCP antibodies are produced by immunizing two different animal species.

20. The kit of claim 17, wherein the capture reagent is immobilized.

21. The kit of claim 20, wherein the kit is an enzyme-linked immunosorbent assay (ELISA).

22. A single-step ELISA assay for detecting and quantifying contaminant host cell proteins in a recombinant protein sample, the method comprising:
a) immobilizing an affinity purified capture reagent comprising anti-host cell protein antibodies, and
b) adding to said immobilized capture reagent:
i) an affinity purified detection reagent comprising anti-host cell protein antibodies and a detectable moeity; and
ii) an aliquot of the recombinant protein sample;
wherein the detection reagent and the recombinant protein sample are added to the immobilized capture reagent substantially simultaneously; and
c) detecting the level of host cell protein in the recombinant protein sample.
